(19) United States
(12) Patent Application Publication
(10) Pub. No.: US 2013/0245690 A1
(43) Pub. Date: Sep. 19, 2013

(54) IMPLANT SYSTEM AND METHOD

(71) Applicants: Keith Bridwell, St. Louis, MO (US); Brian Burd, Memphis, TN (US); Clifford Simmons, Germantown, TN (US)

(72) Inventors: Keith Bridwell, St. Louis, MO (US); Brian Burd, Memphis, TN (US); Clifford Simmons, Germantown, TN (US)

(73) Assignee: WARSAW ORTHOPEDIC, INC., Warsaw, IN (US)

(21) Appl. No.: 13/420,680

(22) Filed: Mar. 15, 2012

(51) Int. Cl. A61B 17/70; A61B 17/88

(52) U.S. Cl. 606/278; 606/279

(57) ABSTRACT

An implant system comprises a fastener including a proximal portion and a distal portion configured for penetrating tissue. The proximal portion defines a first cavity that defines a first longitudinal axis. A connector extends between a first end and a second end configured for disposal in the first cavity and movement within a first plane. The connector is moveable along the first longitudinal axis relative to the distal portion. A receiver is attached to the connector and includes an implant cavity defining a second longitudinal axis and configured for disposal of an implant. The implant cavity is rotatable about the first longitudinal axis such that the implant is rotatable in a second plane relative to the distal portion of the fastener. Methods of use are disclosed.

### TECHNICAL FIELD

The present disclosure generally relates to medical devices for the treatment of musculoskeletal disorders, and more particularly to a spinal implant system and method that employs a connector and provides stabilization of vertebrae, which may include the sacroiliac region.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility. For example, after a disc collapse, severe pain and discomfort can occur due to the pressure exerted on nerves and the spinal column. In another example, disorders of the sacroiliac joint can cause low back and radiating buttock and leg pain in patients.

Non-surgical treatments, such as medication, injection, mobilization, rehabilitation and exercise can be effective, however, may fail to relieve the symptoms associated with these disorders. Surgical treatment of these spinal disorders includes fusion, fixation, discectomy, laminectomy and implantable prosthetics. During surgical treatment, one or more rods may be attached via fasteners to the exterior of two or more vertebral members. Fasteners may also be attached to iliac bone. The present disclosure describes an improvement over these prior art technologies.

### SUMMARY

Accordingly, a surgical system and method is provided. In one particular embodiment, in accordance with the principles of the present disclosure, an implant system comprises a fastener including a proximal portion and a distal portion configured for penetrating tissue. The proximal portion defines a first cavity that defines a first longitudinal axis. A connector extends between a first end and a second end configured for disposal in the first cavity and movement within a first plane. The connector is moveable along the first longitudinal axis relative to the distal portion. A receiver is attached to the connector and includes an implant cavity defining a second longitudinal axis and configured for disposal of an implant. The implant cavity is rotatable about the first longitudinal axis such that the implant is rotatable in a second plane relative to the distal portion of the fastener.

In one embodiment, the implant system comprises a fastener including a proximal portion and a distal portion configured for penetrating tissue. The proximal portion includes an inner surface that defines a first cavity having a substantially triangular configuration. The inner surface includes a first planar portion, a second planar portion and a third arcuate portion. The first cavity defines a first longitudinal axis. A connector has a tapered configuration between a first end and a second end. The first end includes a spline surface radially disposed thereabout and the second end is configured for movable disposal in the first cavity. A receiver includes a spline surface configured to mate with the spline surface of the first end and an implant cavity defining a second longitudinal axis having a transverse orientation relative to first longitudinal axis. The implant cavity is selectively translatable within a coronal plane of a body and rotatable about the second longitudinal axis through an angle of 0 to 360 degrees in a sagittal plane of the body such that the connector is selectively fixable in a position upon fixed engagement of the spline surfaces.

In one embodiment, a method for treating a disorder is provided. The method comprises the steps of providing an implant system comprising: a fastener including a proximal portion and a distal portion configured for penetrating tissue, the proximal portion defining a first cavity that defines a first longitudinal axis, a connector extending between a first end and a second end configured for disposal in the first cavity and movement within a first plane, the connector being moveable along the first longitudinal axis relative to the distal portion, and a receiver attached to the connector and including an implant cavity defining a second longitudinal axis and being configured for disposal of an implant, the implant cavity being rotatable about the first longitudinal axis such that the implant is rotatable in a second plane relative to the distal portion of the fastener; selectively moving the connector in the first plane to a position along the first longitudinal axis; and selectively rotating the implant cavity in the second plane to a position within the second plane.

### BRIEF DESCRIPTION OF THE DRAWINGS

The present disclosure will become more readily apparent from the specific description accompanied by the following drawings, in which:

FIG. 1 is a perspective view of one particular embodiment of an implant system in accordance with the principles of the present disclosure;

FIG. 2 is a side view of a fastener of the implant system shown in FIG. 1;

FIG. 3 is a perspective view of components of the implant system shown in FIG. 1;

FIG. 4 is a top view of the implant system shown in FIG. 1;

FIG. 5 is a perspective view of the components shown in FIG. 1 with parts separated; and

FIG. 6 is a plan view of one embodiment of an implant system in accordance with the principles of the present disclosure attached with vertebrae and an iliac bone of a patient.

Like reference numerals indicate similar parts throughout the figures.

### DETAILED DESCRIPTION

The exemplary embodiments of the implant system and methods disclosed are discussed in terms of medical devices for the treatment of musculoskeletal disorders and more particularly, in terms of an implant system and method for treating a disorder. It is envisioned that the implant system and methods disclosed may provide stability to a portion of the anatomy of a patient, such as, for example, vertebrae, a sacroiliac (SI) joint, iliac bone and maintains structural integrity while reducing stress on the SI joint and/or portions of the anatomy adjacent the SI joint.

In one embodiment, the implant system includes a low profile Iliac wing screw connector and a low profile bone screw. In one embodiment, a screw and rod bolt connector are provided that allow for ilio-sacral implantation. It is contemplated that the screw and connector are low profile such that the screw is a closed head style with a connector that allows for coronal and sagittal adjustment. This configuration enables the screw and connector to be disposed close to a body surface and retain adjustability while maintaining strength of fixation and/or attachment with the body surface.

In one embodiment, a low profile iliac connector and screw are provided that include additional degrees of rotation for an increased level of space with an anatomy and/or body surface.

It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is contemplated that the present disclosure may be employed with other osteal and bone related applications, including those associated with diagnostics and therapeutics. It is further contemplated that the disclosed surgical system and methods may be alternatively employed in a surgical treatment with a patient in a prone or supine position, and/or employ various surgical approaches to the spine, including anterior, posterior, posterior mid-line, lateral, postero-lateral, and/or antero-lateral approaches, and in other body regions. The present disclosure may also be alternatively employed with procedures for treating the lumbar, cervical, thoracic and pelvic regions of a spinal column. The system and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

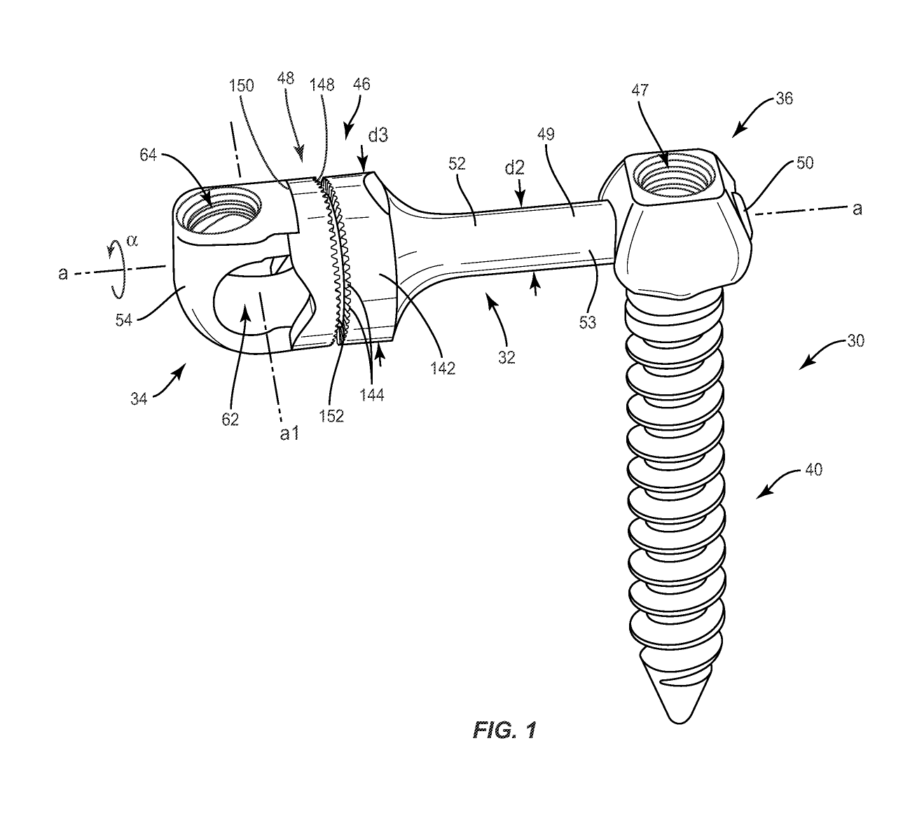

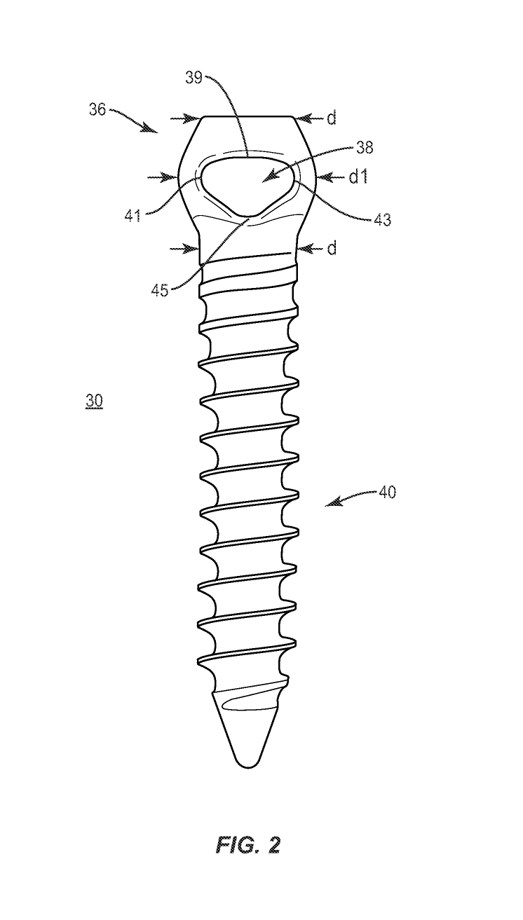

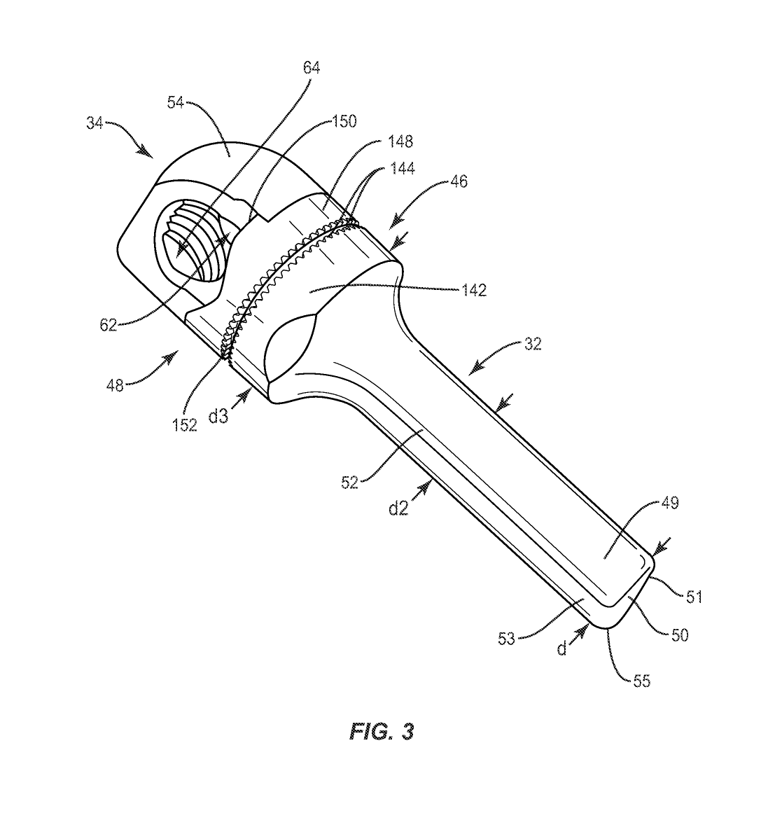

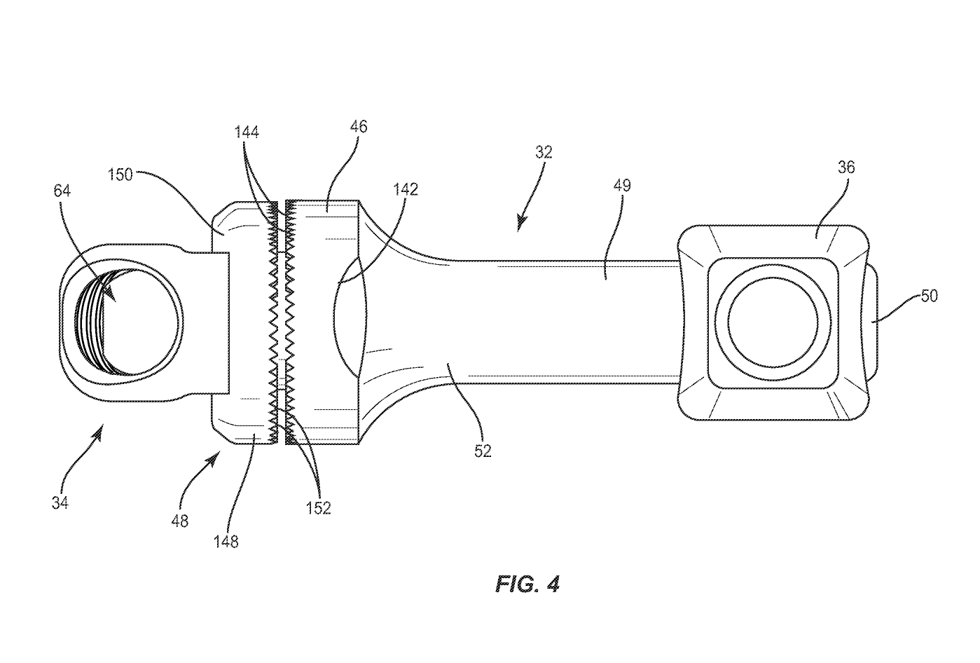

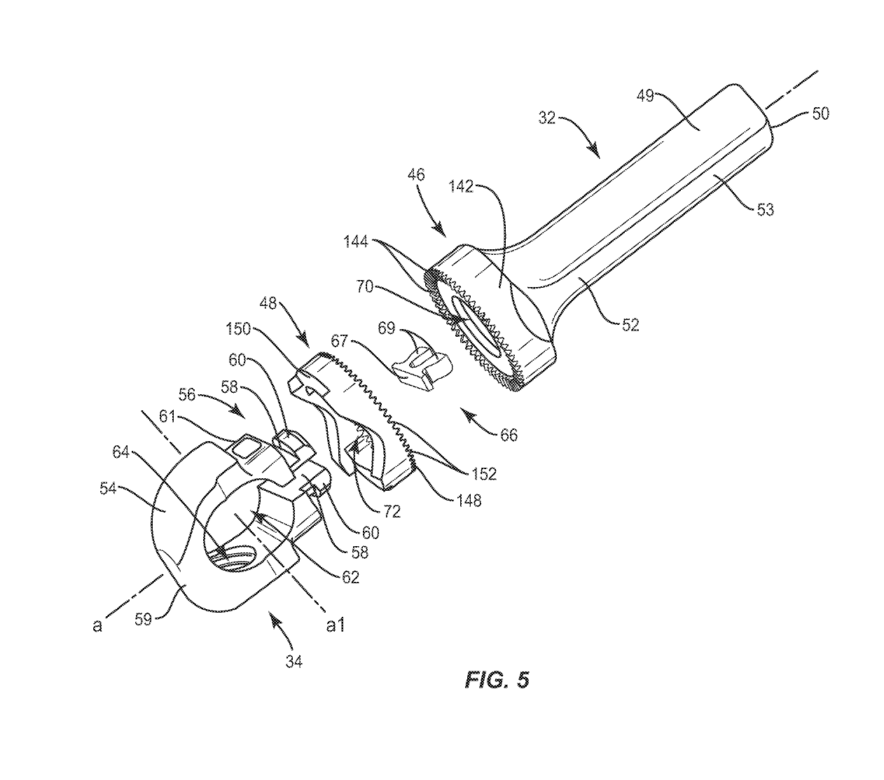

The following discussion includes a description of a surgical system including an implant system, related components and exemplary methods of employing the implant system in accordance with the principles of the present disclosure. Alternate embodiments are also disclosed. Reference will now be made in detail to the exemplary embodiments of the present disclosure, which are illustrated in the accompanying figures. Turning now to FIGS. 1-5, there is illustrated components of a surgical system including an implant system in accordance with the principles of the present disclosure.

The components of the surgical system can be fabricated from biologically acceptable materials suitable for medical applications, including metals, synthetic polymers, ceramics and bone material and/or their composites, depending on the particular application and/or preference of a medical practitioner. For example, the components of the implant system, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of the surgical system may have material composites, including the above materials, to achieve various desired characteristics such as strength, rigidity, elasticity, compliance, biomechanical performance, durability and radiolucency or imaging preference. The components of the surgical system, individually or collectively, may also be fabricated from a heterogeneous material such as a combination of two or more of the above-described materials. The components of the surgical system may be monolithically formed, integrally connected or include fastening elements and/or instruments, as described herein.

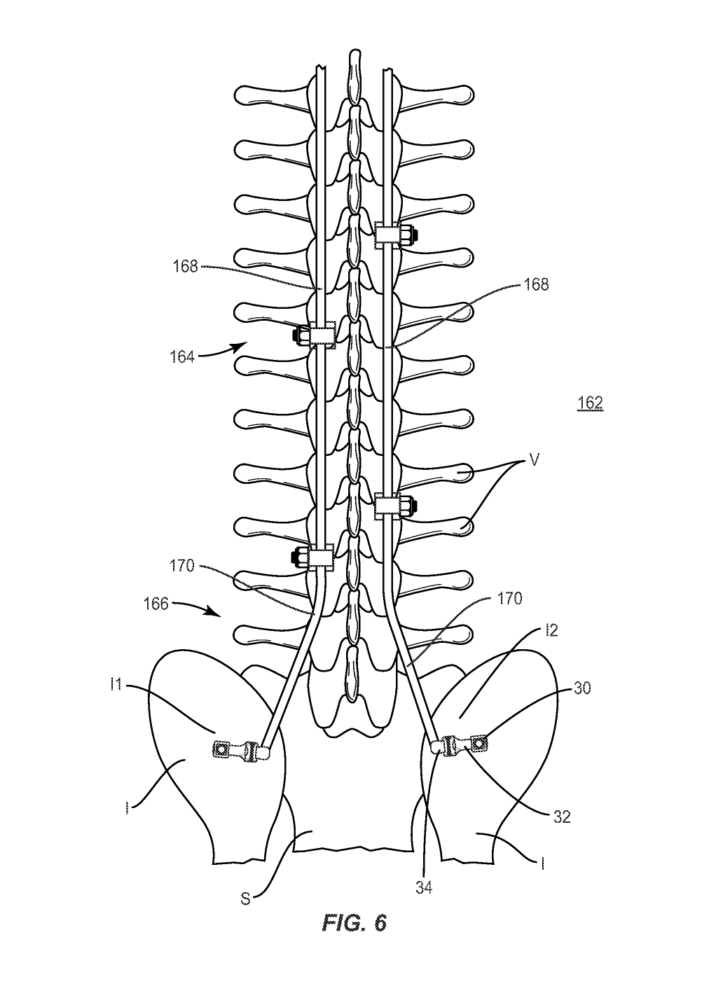

The implant system is configured for attachment to vertebrae and/or iliac bone (as shown, for example, in FIG. 6) during surgical treatment of a spinal disorder, examples of which being discussed herein. The implant system includes a bone fastener 30, a connector 32 and a receiver 34. It is envisioned that the implant system may include one or a plurality of fasteners, connectors and/or receivers.

Bone fastener 30 includes a proximal portion, such as, for example, a head 36 having an inner surface 37 that defines a first cavity, such as, for example, a passageway 38. Passageway 38 defines a first longitudinal axis a. Passageway 38 extends through lateral surfaces of head 36. Passageway 38 is configured to receive and movably support at least a portion of connector 32 such that connector 32 can translate axially within passageway 38 along axis a through a first plane, such as, for example, a coronal plane of a body. It is contemplated that connector 32 may be disposed with bone fastener 30 for relative movement thereto in orientations relative to axis a, such as, for example, transverse, perpendicular and/or other angular orientations such as acute or obtuse, co-axial and/or may be offset or staggered. It is further contemplated that connector 32 may move relative to bone fastener 30 in alternate planes relative to a body, such as, for example, transverse and/or sagittal planes of a body.

Inner surface 37 includes an arcuate portion 39, a first planar portion 41, a second planar portion 43 and an angled portion 45. The portions of inner surface 37 define a substantially triangular configuration of passageway 38. Portion 39 is concavely curved toward a body surface to facilitate capture of connector 32 with the body. It is envisioned that all or only a portion of the passageway 38 may be variously configured and dimensioned, such as, for example, round, oval, oblong, square, rectangular, polygonal, irregular, uniform, non-uniform, offset, staggered, tapered, consistent or variable, depending on the requirements of a particular application. In one embodiment, the first cavity may extend through only a portion of head 36 and not completely through.

Head 36 includes a second threaded cavity 47 configured to receive a coupling member, such as, for example, a setscrew (not shown) to releasably fix connector 32 with bone fastener 30 in a selected position along axis a through the coronal plane of the body. The setscrew is threaded with cavity 47 into fixed engagement with an outer surface of connector. It is envisioned that connector 32 may be coupled with bone fastener 30 in alternate fixation configurations, such as, for example, friction fit, pressure fit, locking protrusion/recess, locking keyway and/or adhesive. It is contemplated that all or only a portion of connector 32 may have alternate surface configurations to enhance fixation with bone fastener 30, such as, for example, rough, arcuate, undulating, mesh, porous, semi-porous, dimpled and/or textured according to the requirements of a particular application.

Head 36 has a reduced thickness having a diameter d and an increased thickness having a diameter d1 to enhance the low profile configuration of bone fastener 30 with a body. As such, head 36 has tapered surfaces such that at least a portion of head 36 may seat more closely with the anatomy of a patient, thereby reducing the profile of bone fastener 30. In one embodiment, at least a portion of the outer surface of head 36 is threaded for penetration with a body surface such that bone fastener 30 has a low profile when fixed with a body of a patient.

Bone fastener 30 includes a distal portion, such as, for example, a shaft 40. Shaft 40 is threaded along the length thereof and configured for penetrating tissue. Shaft 40 has a cylindrical cross section configuration and includes an outer surface having an external thread form. It is contemplated that the thread form may include a single thread turn or a plurality of discrete threads. It is further contemplated that other engaging structures may be located on shaft 40, such as, for example, a nail configuration, barbs, expanding elements, raised elements and/or spikes to facilitate engagement of shaft 40 with tissue, such as, for example, vertebrae and/or iliac bone.

It is envisioned that all or only a portion of shaft 40 may have alternate cross section configurations, such as, for example, oval, oblong, triangular, square, polygonal, irregular, uniform, non-uniform, offset, staggered, undulating, arcuate, variable and/or tapered. It is contemplated that the outer surface of shaft 40 may include one or a plurality of openings. It is further contemplated that all or only a portion of the outer surface of shaft 40 may have alternate surface configurations to enhance fixation with tissue such as, for example, rough, arcuate, undulating, mesh, porous, semi-porous, dimpled and/or textured according to the requirements of a particular application. It is envisioned that all or only a portion of shaft 40 may be disposed at various orientations, relative to axis a, such as, for example, transverse, perpendicular and/or other angular orientations such as acute or obtuse and/or may be offset or staggered. It is further envisioned that all or only a portion of shaft 40 may be cannulated.

Connector 32 extends between a first end 50 and a second end 52. Connector 32 includes an arcuate surface 49, a first planar surface 51, a second planar surface 53 and an angled surface 55, corresponding to the configuration of inner surface 37 of bone fastener 30 for mating engagement therewith. Connector 32 defines a substantially triangular configuration for slidable movement within passageway 38 in a close fitting engagement. It is envisioned that all or only a portion of the cross section configuration of connector 32 may be variously configured and dimensioned, such as, for example, round, oval, oblong, square, rectangular, polygonal, irregular, uniform, non-uniform, offset, staggered, tapered, consistent or variable, depending on the requirements of a particular application.

First end 50 is configured for movement within passageway 38 along axis a in the coronal plane of the body for selective fixation in a position along axis a. Connector 32 is moveable along axis a relative to shaft 40 of fastener 30. It is envisioned that first end 50 may be inserted into passageway 38. Second end 52 is moved within passageway 38 in the coronal plane along axis a, in a first axial direction or a second axial direction oriented in an opposing direction to the first axial direction, by sliding connector 32 relative to head 36. Upon determination of selected positioning of connector 32 relative to shaft 40, which is disposed in tissue, for example, vertebrae and/or iliac bone, the setscrew is threaded with head 36 to fixably engage the outer surface of connector 32.

Connector 32 has a uniform cross section configuration, which defines a diameter d2, extending to second end 52. Second end 52 has a tapered configuration such that second end 52 uniformly tapers to an endmost surface 142 of second end 52 having an increased diameter d3. Diameter d3 provides a limit on axial translation of connector 32 upon engagement with bone fastener 30.

Second end 52 includes a first disk 46, which includes endmost surface 142. Surface 142 is substantially planar and includes an opening 70. Surface 142 includes a splined surface, such as, for example, a plurality of radial splines 144 disposed circumferentially about surface 142. Splines 144 are configured to releasably fix receiver 34 with connector 32 in a selected rotatable position about axis a in a second plane, such as, for example, a sagittal plane of the body relative to shaft 40 of bone fastener 30. It is envisioned that receiver 34 may be coupled with connector 32 in alternate fixation configurations, such as, for example, friction fit, pressure fit, locking protrusion/recess, locking keyway and/or adhesive. It is further envisioned that receiver 34 may be disposed with connector 32 for relative movement thereto in orientations relative to axis a, such as, for example, transverse, perpendicular and/or other angular orientations such as acute or obtuse, co-axial and/or may be offset or staggered. It is contemplated that receiver 34 may move relative to connector 32 in alternate planes relative to a body, such as, for example, transverse and/or coronal planes of a body. It is further contemplated that all or only a portion of surface 142 may have alternate surface configurations to enhance fixation with receiver 34, such as, for example, rough, arcuate, undulating, mesh, porous, semi-porous, dimpled and/or textured according to the requirements of a particular application.

Receiver 34 is selectively rotatable in the sagittal plane of the body and selectively fixable in a position within the sagittal plane. First end 50 defines an opening 70 configured to receive at least a portion of receiver 34 to rotatably connect receiver 34 and connector 32. In one embodiment, receiver 34 may be rotated through an angle of 0 to 360 degrees relative to connector 32.

Receiver 34 includes a body portion 54 having an extension 56 having a reduced thickness configuration for disposal in opening 70. Extension 56 includes a first locking part, such as, for example, prongs 58, which extend transversely from body portion 54 along axis a. Prongs 58 each have a flange 60 that extend laterally. In one embodiment, body portion 54 has an elastic configuration such that prongs 58 are resiliently biased outwardly. It is envisioned that extension 56 may be spring-loaded, or include a biased member, or a shape-memory member.

Extension 56 of receiver 34 is fixed with connector 32 and relative rotation thereto with a second locking part, such as, for example, a wishbone clip 66. Clip 66 has base 64 and extending lobes 69, such that clip 66 has a wishbone configuration. Prongs 58 are inserted within opening 70 such that clip 66 is disposed between prongs 58. Flanges 60 engage an inner surface that defines opening 70 such that prongs 58 retain receiver 34 with connector 32. Clip 66 occupies some or all of the space between prongs 58 to prevent prongs 58 from inward movement to prevent release of flanges 60. This configuration prevents undesired removal of extension 56 from connector 32. In one embodiment, clip 66 may have a width that is approximately equal to or slightly larger than the gap between prongs 58 when prongs 58 and receiver 34 are in an unstressed state. In one embodiment, clip 66 may include a block, such as, for example, a ball, cylinder, planar solid or other relatively solid structure.

Connector 32 is mounted with receiver 34 by inserting extension 56 with opening 70. It is envisioned that prongs 58 can be squeezed together either by external pressure, snap-fit, friction fit and/or threaded engagement.

Receiver 34 includes a second disk 48, which includes endmost surface 148 and an inner surface 150 that defines an opening 72 extending through second disk 48. Surface 148 is substantially planar and includes opening 72, which is configured for disposal of extension 56. Surface 148 includes a splined surface, such as, for example, a plurality of radial splines 152 disposed circumferentially about surface 148. It is contemplated that all or only a portion of surface 142 may have alternate surface configurations to enhance fixation with connector 32, such as, for example, rough, arcuate, undulating, mesh, porous, semi-porous, dimpled and/or textured according to the requirements of a particular application.

Splines 152 are configured to engage splines 142 to releasably fix receiver 34 with connector 32 in a selected rotatable position about axis a in the sagittal plane of the body relative to shaft 40 of bone fastener 30. Splines 142, 152 are configured to mesh such that implant cavity 62 and second disk 48 can rotate and lock at different angles in the sagittal plane. Second disk 48 is locked in position relative to first disk 46 by forcing disks 46, 48 into engagement. In one embodiment, disks 46, 48 are resiliently biased towards for fixed engagement.

Receiver 34 includes an inner surface that defines an implant cavity 62 extending through body portion 54. Implant cavity 62 is configured for disposal of an implant, such as, for example, a vertebral rod of a vertebral rod system a surgical arthrodesis procedure 162, described with regard to FIG. 6, and defines a second longitudinal axis a1. Implant cavity 62 has a transverse orientation relative to passageway 38. Axis a is transversely orientated relative to axis a1. Implant cavity 62 is rotatable relative to axis a1, through an angle α, such that implant cavity 62 is rotatable in the sagittal plane of the body relative to shaft 40 of bone fastener 30. It is contemplated that angle α may be in a range of 0 to 360 degrees relative to axis a1.

It is envisioned that implant cavity 62 may be variously configured and dimensioned, such as, for example, round, oval, oblong, square, rectangular, polygonal, irregular, uniform, non-uniform, offset, staggered, tapered, consistent or variable, depending on the requirements of a particular application. It is further envisioned that implant cavity 62 may be movable relative to axis a1 in orientations, such as, for example, transverse, perpendicular and/or other angular orientations such as acute or obtuse, co-axial and/or may be offset or staggered. It is contemplated that implant cavity 62 may move relative to connector 32 in alternate planes relative to a body, such as, for example, transverse and/or coronal planes of a body. In one embodiment, implant cavity 62 may be disposed at an angle of about 30 to about 150 degrees relative to passageway 38 and axis a may be disposed at an angle of about 30 to about 150 degrees relative to axis a1.

Body portion 54 includes a threaded cavity 64 configured to receive a coupling member, such as, for example, a setscrew (not shown) to releasably fix an implant, such as, for example, a vertebral rod of a vertebral rod system 162 (FIG. 6) with receiver 34 in a selected rotation position at an angle α relative to axis a1 in the sagittal plane of the body relative to shaft 40 of bone fastener 30. The setscrew is threaded with cavity 64 into fixed engagement with an outer surface of the rod implant. It is envisioned that rod 162 may be coupled with receiver 34 in alternate fixation configurations, such as, for example, friction fit, pressure fit, locking protrusion/recess, locking keyway and/or adhesive. It is contemplated that all or only a portion of receiver 34 may have alternate surface configurations to enhance fixation with the rod implant, such as, for example, rough, arcuate, undulating, mesh, porous, semi-porous, dimpled and/or textured according to the requirements of a particular application.

In assembly, operation and use, the implant system including bone fastener 30, connector 32 and receiver 34 is employed with a surgical procedure for treatment of a spinal disorder affecting a section of a spine and/or ilium bones of a pelvis of a patient, as discussed herein. The implant system may also be employed with other surgical procedures. The implant system is employed with a surgical procedure for treatment of a condition or injury of an affected section of the spine including vertebrae V, which may include sacrum S, and/or ilium I, as shown in FIG. 6. It is contemplated that the implant system including bone fastener 30, connector 32 and receiver 34 is attached to vertebrae V and/or ilium I for a surgical arthrodesis procedure, such as fusion, and/or dynamic stabilization application of the affected section of the spine to facilitate healing and therapeutic treatment, while providing flexion, extension and/or torsion capability. In fusion applications, it is contemplated that the implant system provides flexibility to a bone construct for improved compliance and less rigidity. In dynamic applications, it is contemplated that the implant system provides flexibility to a bone construct.

In use, to treat the affected section of the spine and/or ilium bones of a pelvis, a medical practitioner obtains access to a surgical site including vertebra V and/or ilium I in any appropriate manner, such as through incision and retraction of tissues. It is envisioned that the implant system including bone fastener 30, connector 32 and receiver 34 may be used in any existing surgical method or technique including open surgery, mini-open surgery, minimally invasive surgery and percutaneous surgical implantation, whereby the vertebrae V and/or ilium I is accessed through a micro-incision, or sleeve that provides a protected passageway to the area. Once access to the surgical site is obtained, the particular surgical procedure is performed for treating the bone disorder. The implant system including bone fastener 30, connector 32 and receiver 34 is then employed to augment the surgical treatment. The implant system including bone fastener 30, connector 32 and receiver 34 can be delivered or implanted as a pre-assembled device or can be assembled in situ. The implant system may be completely or partially revised, removed or replaced, for example, replacing a rod implant of vertebral rod system 162 and/or one or all of the components of the implant system.

In one embodiment, vertebral rod system 162 includes one or a plurality of vertebral rods and fasteners for attaching the rods to vertebrae V, as shown in FIG. 6. System 162 extends from a first portion 164 to a second portion 166 disposed adjacent a sacroiliac (SI) region SIR of the patient. Second portion includes two axially aligned and spaced apart rods 168. Rods 168 each have a rigid, arcuate portion 170 extending across a sacrum S and ilium I of region SIR.

A first bone fastener 30 is configured for fixation with an ilium surface I1 and a second bone fastener 30 is configured for fixation with an ilium surface 12. Pilot holes are made in ilium surfaces I1, I2 for receiving first and second bone fasteners 30. Each threaded shaft 40 of first and second bone fasteners 30 are inserted or otherwise connected to ilium surfaces I1, I2, according to the particular requirements of the surgical treatment. Connector 32 is attached with bone fastener 30, and receiver 34 is attached with connector 32, as described above.

According to the orientation and position of each portion 170, each second end 52 is selectively moved within passageway 38 in a coronal plane of the patient along axis a by sliding connector 32 relative to head 36. Upon determination of selected positioning of connector 32 relative to shaft 40, the setscrew is threaded with head 36 to fixably engage the outer surface of connector 32 to lock connector 32 in the selected axial position relative to bone fastener 30.

Implant cavity 62 is selectively rotated to an angle α relative to axis a1 in the sagittal plane of the patient corresponding to the orientation and position of each portion 170. This configuration allows orientation of implant cavity 62 to receive each portion 170 for disposal of each portion 170 therein. The setscrew is threaded with cavity 64 into fixed engagement with an outer surface of each portion 170 to fix receiver 34 in the selected rotation orientation, for example, at angle α relative to axis a1 in the sagittal plane, relative to bone fastener 30. The splined surfaces of disks 46, 48 are brought into fixed engagement, as discussed above, to lock receiver 34 in the selected rotation orientation.

It is contemplated that the implant system configuration of bone fastener 30, connector 32 and receiver 34 are low profile ilium surfaces I1, I2 that allows for coronal and sagittal adjustment. This configuration enables the implant system to be disposed close to a body surface and retain adjustability while maintaining strength of fixation and/or attachment with the body surface.

In one embodiment, the implant system includes an agent, which may be disposed, packed or layered within, on or about the components and/or surfaces of the implant system. It is envisioned that the agent may include bone growth promoting material, such as, for example, bone graft to enhance fixation of the fixation elements with vertebrae V.

It is contemplated that the agent may include therapeutic polynucleotides or polypeptides. It is further contemplated that the agent may include biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as HA, calcium phosphate and calcium sulfite, biologically active agents, for example, gradual release compositions such as by blending in a bioresorbable polymer that releases the biologically active agent or agents in an appropriate time dependent fashion as the polymer degrades within the patient. Suitable biologically active agents include, for example, BMP, Growth and Differentiation Factors proteins (GDF) and cytokines. The components of the implant system can be made of radiolucent materials such as polymers. Radiomarkers may be included for identification under x-ray, fluoroscopy, CT or other imaging techniques. It is envisioned that the agent may include one or a plurality of therapeutic agents and/or pharmacological agents for release, including sustained release, to treat, for example, pain, inflammation and degeneration.

It is envisioned that the use of microsurgical and image guided technologies may be employed to access, view and repair spinal deterioration or damage, with the aid of the implant system. Upon completion of the procedure, the surgical instruments and assemblies are removed and the incision is closed.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

1. An implant system comprising:
a fastener including a proximal portion and a distal portion configured for penetrating tissue, the proximal portion defining a first cavity that defines a first longitudinal axis;
a connector extending between a first end and a second end configured for disposal in the first cavity and movement within a first plane, the connector being moveable along the first longitudinal axis relative to the distal portion;
a receiver attached to the connector and including an implant cavity defining a second longitudinal axis and being configured for disposal of an implant, the implant cavity being rotatable about the first longitudinal axis such that the implant is rotatable in a second plane relative to the distal portion of the fastener; and
wherein the connector includes a first disk having a first surface that includes a splined configuration, and the receiver includes a second disk having a first surface that includes a splined configuration that mates with the splined configuration of the first disk.

2. The implant system of claim 1, wherein the first plane is a coronal plane of a body and the second plane is a sagittal plane of the body.

3. The implant system of claim 1, wherein the connector is selectively moveable in the first plane for selective fixation in a position along the first longitudinal axis.

4. The implant system of claim 1, wherein the receiver is selectively rotatable in the second plane for selective fixation in a position within the second plane.

5. (canceled)

6. The implant system of claim 1, wherein the splined configuration of the first surface of the first disk and the splined configuration of the first surface of the second disk are radial splines.

7. The implant system of claim 1, wherein the connector includes an extension having a reduced thickness for disposal within the first cavity.

8. The implant system of claim 1, wherein the receiver includes a first locking part and the connector includes a second locking part, the first locking part being engageable with the second locking part to lock the receiver with the connector.

9. The implant system of claim 8, wherein the second locking part has a wishbone configuration.

10. The implant system of claim 1, wherein the first cavity has a transverse orientation relative to the implant cavity.

11. The implant system of claim 1, wherein the first longitudinal axis has a transverse orientation relative to the second longitudinal axis.

12. The implant system of claim 1, wherein the connector includes a first disk and the receiver includes a second disk that mates with the second disk for fixed engagement therebetween.

13. The implant system of claim 1, wherein the implant cavity is rotatable through an angle of 0 to 360 degrees.

14. The implant system of claim 1, wherein the connector has a tapered configuration from the first end to the second end.

15. The implant system of claim 1, wherein the proximal portion of the fastener includes an inner surface that defines the first cavity having a substantially triangular configuration.

16. The implant system of claim 1, wherein the proximal portion of the fastener includes an inner surface that includes a first planar portion, a second planar portion and a third arcuate portion.

17. An implant system comprising:
a fastener including a proximal portion and a distal portion configured for penetrating tissue, the proximal portion including an inner surface that defines a first cavity having a substantially triangular configuration such that the inner surface includes a first planar portion, a second planar portion and a third arcuate portion, the first cavity defining a first longitudinal axis;
a connector having a tapered configuration between a first end and a second end, the first end including a spline surface radially disposed thereabout and the second end being configured for movable disposal in the first cavity; and
a receiver including a spline surface configured to mate with the spline surface of the first end and an implant cavity defining a second longitudinal axis having a transverse orientation relative to first longitudinal axis, the implant cavity being selectively translatable within a coronal plane of a body and rotatable about the second longitudinal axis through an angle of 0 to 360 degrees in a sagittal plane of the body such that the connector is selective fixable in a position upon fixed engagement of the spline surfaces.

18. A method for treating a disorder, comprising the steps of:
providing an implant system comprising:
a fastener including a proximal portion and a distal portion configured for penetrating tissue, the proximal portion defining a first cavity that defines a first longitudinal axis,
a connector extending between a first end and a second end configured for disposal in the first cavity and movement within a first plane, the connector being moveable along the first longitudinal axis relative to the distal portion, and
a receiver attached to the connector and including an implant cavity defining a second longitudinal axis and being configured for disposal of an implant, the implant cavity being rotatable about the first longitudinal axis such that the implant is rotatable in a second plane relative to the distal portion of the fastener;
wherein the connector includes a first disk having a first surface that includes a splined configuration, and the receiver includes a second disk having a first surface that includes a splined configuration that mates with the splined configuration of the first disk;
selectively moving the connector in the first plane to a position along the first longitudinal axis; and
selectively rotating the implant cavity in the second plane to a position within the second plane.

19. The method of claim 18, further comprising the step of engaging the connector with the fastener to fix the implant cavity in the position along the first longitudinal axis and the position within the second plane.

20. The method of claim 18, wherein the step of selectively rotating the implant cavity includes rotating the implant cavity through an angle in a range between 0 to 360 degrees.

21. The implant system of claim 1, wherein a setscrew is configured to releasably fix the connector to the proximal portion of the fastener.
